(19) United States
(12) Patent Application Publication
(10) Pub. No.: US 2022/0207730 A1
(43) Pub. Date: Jun. 30, 2022

(54) Systems and Methods for Automated Image Analysis

(71) Applicant: The Regents of the University of California, Oakland, CA (US)

(72) Inventors: Corey Arnold, Oakland, CA (US); Jiayun Li, Oakland, CA (US); William Speier, Oakland, CA (US); Wenyun Li, Oakland, CA (US)

(21) Appl. No.: 17/612,062

(22) PCT Filed: May 26, 2020
(86) PCT No.: PCT/US2020/034552

§ 371 Date: Nov. 17, 2021

Related U.S. Application Data
(60) Provisional application No. 62/852,625, filed on May 24, 2019.

(51) Int. Cl. G06T 7/00; G16H 30/20; G16H 15/00

(52) U.S. Cl. CPC G06T 7/0012; G16H 30/20; G06T 2207/20081; G06T 2207/30024; G06T 2207/20084; G16H 15/00

(57) ABSTRACT

In accordance with one aspect of the disclosure, an image analysis system is provided. The image analysis system includes at least one processor configured to access image tiles associated with a patient, each tile comprising a portion of a whole slide image, individually provide a first group of image tiles to a first trained model, receive a first set of feature objects from the first trained model, cluster feature objects from the first set of feature objects to form a number of clusters, calculate a number of attention scores based on the first set of feature objects, select a second group of tiles, individually provide the second group of image tiles to a second trained model, receive a second set of feature objects from the second trained model, generate a cancer grade indicator, and cause the cancer grade indicator to be output.

### CROSS-REFERENCE TO RELATED APPLICATIONS

This application is based on, claims the benefit of, and claims priority to U.S. Provisional Application No. 62/852,625, filed May 24, 2019, which is hereby incorporated by reference herein in its entirety for all purposes.

### STATEMENT REGARDING FEDERALLY SPONSORED RESEARCH

This invention was made with government support under Grant Number CA220352, awarded by the National Institutes of Health. The government has certain rights in the invention.

### BACKGROUND OF THE INVENTION

Medical imaging is a key tool in the practice of modern clinical medicine. Imaging is used in an extremely broad array of clinical situations, from diagnosis to delivery of therapeutics to guiding surgical procedures. While medical imaging provides an invaluable resource, it also consumes extensive resources. Furthermore, imaging systems require extensive human interaction to setup and operate, and then to analyze the images and make clinical decisions.

As just one clinical example, prostate cancer is the most common and second deadliest cancer in men in the U. S, accounting for nearly 1 in 5 new cancer diagnoses. Gleason grading of biopsied tissue is a key component in patient management and treatment selection. The Gleason score (GS) is determined by the two most prevalent Gleason patterns in the tissue section. Gleason patterns range from 1 (G1), representing tissue that is close to normal glands, to 5 (G5), indicating more aggressive cancer. Patients with high risk cancer (i.e., GS>7 or G4+G3) are usually treated with radiation, hor-monal therapy, or radical prostatectomy, while those with low- to intermediate-risk prostate cancer (i.e., GS<6 or G3+G4) are candidates for active surveillance.

Currently, pathologists need to scan through a histology slide, searching for relevant regions on which to ascertain Gleason scores. This process can be time-consuming and prone to observer variability. Additionally, there are many unique challenges in developing computer aided diagnosis (CAD) tools for whole slide images (WSIs), such as the very large image size, the heterogeneity of slide contents, the insufficiency of fine-grained labels, and possible artifacts caused by pen markers and stain variations.

It would therefore be desirable to provide systems and methods that increase the clinical utility of medical imaging.

### SUMMARY OF THE INVENTION

The present disclosure provides systems and methods that overcome the aforementioned drawbacks by providing new systems and methods for processing and analyzing medical images. The systems and methods provided herein can be utilized to reduce the total investment of human time required for medical imaging applications. In one non-limiting example, systems and methods are provided for automatically analyzing images, for example, such as whole slide images (e.g., digital images of biopsy slides).

In accordance with one aspect of the disclosure, an image analysis system is provided. The image analysis system includes a storage system configured to have image tiles stored therein, at least one processor configured to access the storage system and configured to access image tiles associated with a patient, each tile comprising a portion of a whole slide image, individually provide a first group of image tiles to a first trained model, each image tile included in the first group of image tiles having a first magnification level, receive a first set of feature objects from the first trained model in response to providing the first group of image tiles to the first trained model, cluster feature objects from the first set of feature objects to form a number of clusters, calculate a number of attention scores based on the first set of feature objects, each attention score being associated with an image tile included in the first group of image tiles, select a second group of tiles from the number of image tiles based on the clusters and the attention scores, each image tile included in the second group of image tiles having a second magnification level, individually provide the second group of image tiles to a second trained model, receive a second set of feature objects from the second trained model in response to providing the second group of image tiles to the second trained model, generate a cancer grade indicator based on the second set of feature objects from the second trained model, and cause the cancer grade indicator to be output to at least one of a memory or a display.

In accordance with another aspect of the disclosure, an image analysis method is provided. The image analysis method includes receiving pathology image tiles associated with a patient, each tile comprising a portion of a whole pathology slide, providing a first group of image tiles to a first trained learning network, each image tile included in the first group of image tiles having a first magnification level, receiving first feature objects from the first trained learning network, clustering the first feature objects to form a number of clusters, calculating a number of attention scores based on the first feature objects, each attention score being associated with an image tile included in the first group of image tiles, selecting a second group of tiles from the number of image tiles based on the clusters and the attention scores, each image tile included in the second group of image tiles having a second magnification level that differs from the first magnification level, providing the second group of image tiles to a second trained learning network, receiving second feature objects from the second trained learning network, generating a cancer grade indicator based on the second feature objects from the second trained learning network, and outputting the cancer grade indicator to at least one of a memory or a display.

In accordance with yet another aspect of the disclosure, a whole slide image analysis method is provided. The whole slide image analysis method includes operating an imaging system to form image tiles associated with a patient, each tile comprising a portion of a whole slide image, individually providing a group of image tiles to a first trained model, each image tile included in the first group of image tiles having a first magnification level, receiving a first set of feature objects from the first trained model, grouping feature objects in the first set of features objects based on clustering criteria, calculating a number of attention scores based on the feature objects, each attention score being associated with an image tile included in the first group of image tiles, selecting a second group of tiles from the image tiles based on grouping of the feature objects and the attention scores, each image tile included in the second group of image tiles having a second magnification level that differs from the first magnification level, providing the second group of image tiles to a second trained model, receiving a second set of feature objects from the second trained model, generating a cancer grade indicator based on the second set of feature objects, generating a report based on the cancer grade indicator, and causing the report to be output to at least one of the memory or the display.

The foregoing and other aspects and advantages of the invention will appear from the following description. In the description, reference is made to the accompanying drawings which form a part hereof, and in which there is shown by way of illustration configurations of the invention. Any such configuration does not necessarily represent the full scope of the invention, however, and reference is made therefore to the claims and herein for interpreting the scope of the invention.

### BRIEF DESCRIPTION OF THE DRAWINGS

FIG. 1 is an example of an image analysis system in accordance with the disclosed subject matter.

FIG. 2 is an example of hardware that can be used to implement a computing device and a supplemental computing device shown in FIG. 1 in accordance with the disclosed subject matter.

FIG. 3 is an example of a flow for generating one or more metrics related to the presence of cancer in a patient.

FIG. 4 is an exemplary process for training a first stage model and a second stage model.

FIG. 5 is an exemplary process for generating cancer predictions for a patient.

FIG. 6 is a Confusion matrix for Gleason grade classification on a test set.

FIG. 7 is an example of a flow for generating one or more metrics related to the presence of cancer in a patient.

FIG. 8 is an exemplary process for training a first stage model and a second stage model.

FIG. 9 is an exemplary process for generating cancer predictions for a patient.

FIG. 10A is a graph of ROC curves for the detection stage cancer models trained at 5×.

FIG. 10B is a graph of PR curves for the detection stage cancer models trained at 5×.

FIG. 11 is a confusion matrix for the MRMIL model on GG prediction.

### DETAILED DESCRIPTION

The present disclosure provides systems and methods that can reduce human and/or trained clinician time required to analyze medical images. As one non-limiting example, the present disclosure provides example of the inventive concepts provided herein applied to the analysis of images such as brightfield images, however, other imaging modalities beyond brightfield imaging and applications within each modality are contemplated, such as fluorescent imaging, fluorescence in situ hybridization (FISH) imaging, and the like. In the non-limiting example of brightfield images, the systems and methods provided herein can determine a grade of cancer and/or cancerous regions in a whole slide image (e.g., a digital image of a biopsy slide).

In some configurations of the present disclosure, an attention-based multiple instance learning (MIL) model is provided that can predict slide-level labels, but also provide visualization of relevant regions using inherent attention maps. Unlike previous work that relied on labor intensive labels, such as manually drawn regions of interest (ROIs) around glands, our model is trained using labels, such as slide-level labels, also known as weak labels, which can be easily retrieved from pathology reports. In some configurations, a two stage model is provided that detects suspicious regions at a lower resolution (e.g. 5×), and further analyzes the suspicious regions at a higher resolution (e.g. 10×), which is similar to pathologists' diagnostic process. The model was trained and validated on a dataset of 2,661 biopsy slides from 491 patients. The model achieved state-of-the-art performance, with a classification accuracy of 85.11% on a hold-out test set consisting of 860 slides from 227 patients. ROI-level classification

Early work on WSI analysis mainly focused on classifying small ROIs, which usually were selected by pathologists from the large tissue slide. However, this does not accurately reflect the true clinical task as to ensure completeness, pathologists must grade the entire tissue section rather than sub-selected representative ROIs. This makes models based on ROIs un-suitable for automated Gleason grading. Slide-level classification

Instead of relying on ROIs, more recent research has focused on slide-level classification. One group developed a two-stage Gleason classification model. In the first-stage, a tile-level classifier was trained with over 112 million annotated tiles from prostatectomy slides. In the second stage, predictions from the first stage were summarized to a K-nearest neighbor classifier for Gleason scoring. They achieved an average accuracy of 70% in four-class Gleason group classification (1, 2, 3, or 4-5). However, these methods required a well-trained tile-level classifier, which can only be developed on a dataset with manually drawn ROIs or slides with homo-geneous tissue contents. Moreover, they did not incorporate information embedded in slide-level labels.

To address these challenges, previous work has proposed using an MIL framework for WSI classification, where the slide was represented as a bag and tiles within the bag were modeled as instances in the bag. MIL models can be roughly divided into two types instance-based and bag-based. Bag-based methods project instance features into low-dimensional rep-resentations and often demonstrate superior performance for bag-level classification tasks. However, as bag-level methods lack the ability to predict instance-level labels, they are less interpretable and thus sub-optimal for problems where obtaining instance labels is important. One group proposed an attention-based deep learning model that can achieve comparable perfor-mances to bag-level models without losing interpretability. A low-dimensional instance embedding, an attention mech-anism for aggregating instance-level features, and a final bag-level classifier were all parameterized with a neural net-work. They applied the model on two histology datasets consisting of small tiles extracted from WSis and demon-strated promising performance. However, they did not apply the model on larger and more heterogeneous WSis. Also, attention maps were only used for a visualization method.

Another group applied an instance-level MIL model for binary prostate biopsy slide classification (i.e. cancer versus non-cancer). Their model was developed on a large dataset consisting of 12,160 biopsy slides, and achieved over 95% area under the curve of the receiver operating characteristic (AUROC). Yet, they did not address the more difficult grading problem. Unlike previous model, the model provided herein improves the attention mechanism with instance dropout. Instead of only using the attention map for visualization, the model provided herein may utilize it to automatically localize informative areas, which then get analyzed at higher resolution for cancer grading.

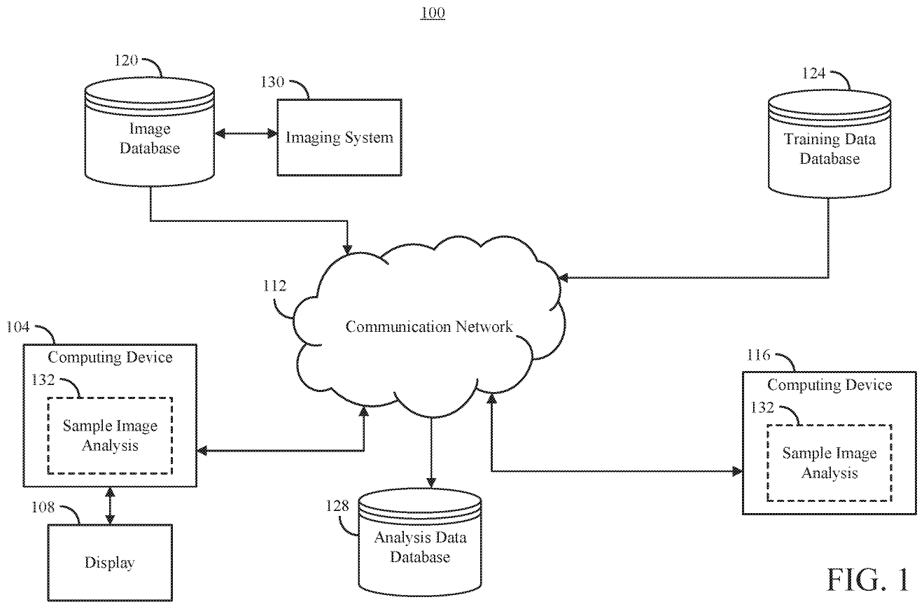

FIG. 1 shows an example of an image analysis system 100 in accordance with some aspects of the disclosed subject matter. In some configurations, the image analysis system 100 can include a computing device 104, a display 108, a communication network 112, a supplemental computing device 116, an image database 120, a training data database 124, and an analysis data database 128. The computing device 104 can be in communication (e.g., wired communication, wireless communication) with the display 108, the supplemental computing device 116, the image database 120, the training data database 124, and the analysis data database 128. The image database 120 is created from data or images derived from an imaging system 130. The imaging system 130 may be a pathology system, a digital pathology system, or an in-vivo imaging system. 100311 The computing device 104 can implement portions of an image analysis application 132, which can involve the computing device 104 transmitting and/or receiving instructions, data, commands, etc. from one or more other devices. For example, the computing device 104 can receive image data from the image database 120, receive training data from the training data database 124, and/or transmit reports and/or raw data generated by the image analysis application 132 to the display 108 and/or the analysis data database 128.

The supplementary computing device 116 can implement portions of the image analysis application 132. It is understood that the image analysis system 100 can implement the image analysis application 132 without the supplemental computing device 116. In some aspects, the computing device 104 can cause the supplemental computing device 116 to receive image data from the image database 120, receive training data from the training data database 124, and/or transmit reports and/or raw data generated by the image analysis application 132 to the display 108 and/or the analysis data database 128. In this way, a majority of the image analysis application 132 can be implemented by the supplementary computing device 116, which can allow a larger range of devices to be used as the computing device 104 because the required processing power of the computing device 104 may be reduced.

The image database 120 can include image data. In one non-limiting example, the images may include images of a biopsy slide associated with a patient (e.g., a whole slide image). The biopsy slide can include tissue taken from a region of the patient such as the prostate, the liver, one or both of the lungs, etc. The image data can include a number of slide images associated with a patient. In some aspects, multiple slide images can be associated with a single patient. For example, a first slide image and a second slide image can be associated with a target patient.

The training data database 124 can include training data that the image analysis application 132 can use to train one or more machine learning models including networks such as convolutional neural networks (CNNs). More specifically, the training data can include weakly annotated training images (e.g., slide-level annotations) that can be used to train one or more machine learning models using a learning process such as a semi-supervised learning process. The training data will be discussed in further detail below.

The image analysis application 132 can automatically generate one or more metrics related to a cancer (e.g., prostate cancer) based on an image. For example, the image analysis application 132 can automatically generate an indication of whether or not a patient has cancer (e.g., either a “yes” or “no” categorization), a cancer grade (e.g., benign, low grade, high grade, etc.), regions of the image (and by extension, the biopsy tissue) that are most cancerous and/or relevant, and/or other cancer metrics. In some configurations, low-grade can include Gleason grade 3, and high-grade can include Gleason grade 4 and Gleason grade 5.

The image analysis application 132 can also automatically generate one or more reports based on the indication of whether or not the patient has cancer, the cancer grade, the regions of the image that are most cancerous and/or relevant, and/or other cancer metrics, as well as the image. The image analysis application 132 can output one or more of the cancer metrics and/or reports to the display 108 (e.g., in order to display the cancer metrics and/or reports to a medical practitioner) and/or to a memory, such as a memory included in the analysis data database 128 (e.g., in order to store the cancer metrics and/or reports).

As shown in FIG. 1, the communication network 112 can facilitate communication between the computing device 104, the supplemental computing device 116, the image database 120, the training data database 124, and the analysis data database 128. In some configurations, the communication network 112 can be any suitable communication network or combination of communication networks. For example, the communication network 112 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some configurations, the communication network 112 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and the like.

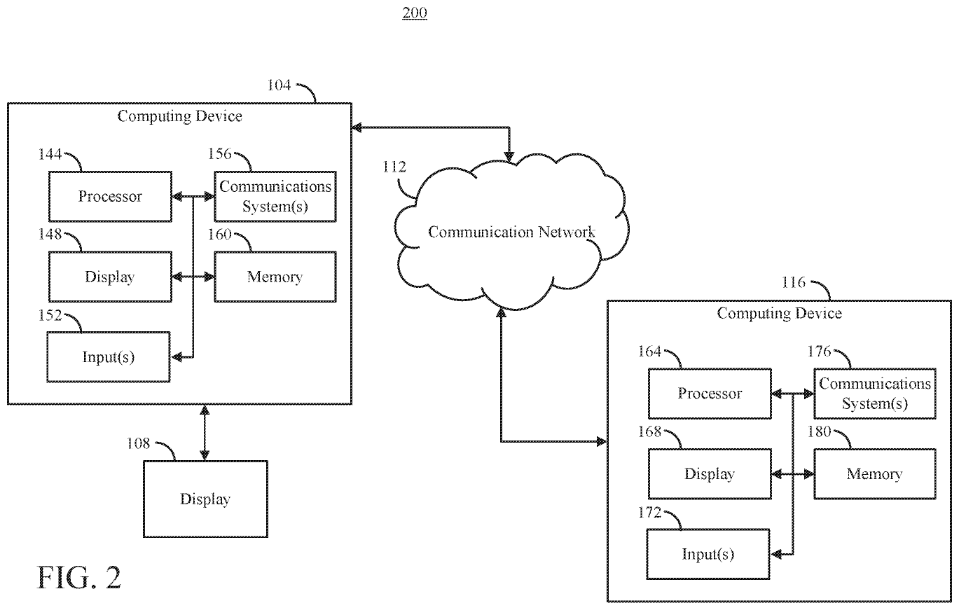

FIG. 2 shows an example of hardware that can be used to implement a computing device 104 and a supplemental computing device 116 shown in FIG. 1 in accordance with some aspects of the disclosed subject matter. As shown in FIG. 2, the computing device 104 can include a processor 144, a display 148, an input 152, a communication system 156, and a memory 160. The processor 144 can implement at least a portion of the image analysis application 132, which can, for example, be executed from a program (e.g., saved and retrieved from the memory 160). The processor 144 can be any suitable hardware processor or combination of processors, such as a central processing unit (“CPU”), a graphics processing unit (“GPU”), etc., which can execute a program, which can include the processes described below.

In some configurations, the display 148 can present a graphical user interface. In some configurations, the display 148 can be implemented using any suitable display devices, such as a computer monitor, a touchscreen, a television, etc. In some configurations, the inputs 152 of the computing device 104 can include indicators, sensors, actuatable buttons, a keyboard, a mouse, a graphical user interface, a touch-screen display, etc. In some configurations, the inputs 152 can allow a user (e.g., a medical practitioner, such as an oncologist) to interact with the computing device 104, and thereby to interact with the supplemental computing device 116 (e.g., via the communication network 112). The display 108 can be a display device such as a computer monitor, a touchscreen, a television, and the like.

In some configurations, the communication system 156 can include any suitable hardware, firmware, and/or software for communicating with the other systems, over any suitable communication networks. For example, the communication system 156 can include one or more transceivers, one or more communication chips and/or chip sets, etc. In a more particular example, the communication system 156 can include hardware, firmware, and/or software that can be used to establish a coaxial connection, a fiber optic connection, an Ethernet connection, a USB connection, a Wi-Fi connection, a Bluetooth connection, a cellular connection, etc. In some configurations, the communication system 156 allows the computing device 104 to communicate with the supplemental computing device 116 (e.g., directly, or indirectly such as via the communication network 112).

In some configurations, the memory 160 can include any suitable storage device or devices that can be used to store instructions, values, etc., that can be used, for example, by the processor 144 to present content using the display 148 and/or the display 108, to communicate with the supplemental computing device 116 via communications system(s) 156, etc. The memory 160 can include any suitable volatile memory, non-volatile memory, storage, or any suitable combination thereof. For example, the memory 160 can include RAM, ROM, EEPROM, one or more flash drives, one or more hard disks, one or more solid state drives, one or more optical drives, etc. In some configurations, the memory 160 can have encoded thereon a computer program for controlling operation of the computing device 104 (or the supplemental computing device 116). In such configurations, the processor 144 can execute at least a portion of the computer program to present content (e.g., user interfaces, images, graphics, tables, reports, and the like), receive content from the supplemental computing device 116, transmit information to the supplemental computing device 116, and the like.

Still referring to FIG. 2, the supplemental computing device 116 can include a processor 164, a display 168, an input 172, a communication system 176, and a memory 180. The processor 164 can implement at least a portion of the image analysis application 132, which can, for example, be executed from a program (e.g., saved and retrieved from the memory 180). The processor 164 can be any suitable hardware processor or combination of processors, such as a central processing unit (CPU), a graphics processing unit (GPU), and the like, which can execute a program, which can include the processes described below.

In some configurations, the display 168 can present a graphical user interface. In some configurations, the display 168 can be implemented using any suitable display devices, such as a computer monitor, a touchscreen, a television, etc. In some configurations, the inputs 172 of the supplemental computing device 116 can include indicators, sensors, actuatable buttons, a keyboard, a mouse, a graphical user interface, a touch-screen display, etc. In some configurations, the inputs 172 can allow a user (e.g., a medical practitioner, such as an oncologist) to interact with the supplemental computing device 116, and thereby to interact with the computing device 104 (e.g., via the communication network 112).

In some configurations, the communication system 176 can include any suitable hardware, firmware, and/or software for communicating with the other systems, over any suitable communication networks. For example, the communication system 176 can include one or more transceivers, one or more communication chips and/or chip sets, etc. In a more particular example, the communication system 176 can include hardware, firmware, and/or software that can be used to establish a coaxial connection, a fiber optic connection, an Ethernet connection, a USB connection, a Wi-Fi connection, a Bluetooth connection, a cellular connection, and the like. In some configurations, the communication system 176 allows the supplemental computing device 116 to communicate with the computing device 104 (e.g., directly, or indirectly such as via the communication network 112).

In some configurations, the memory 180 can include any suitable storage device or devices that can be used to store instructions, values, and the like, that can be used, for example, by the processor 164 to present content using the display 168 and/or the display 108, to communicate with the computing device 104 via communications system(s) 176, and the like. The memory 180 can include any suitable volatile memory, non-volatile memory, storage, or any suitable combination thereof. For example, the memory 180 can include RAM, ROM, EEPROM, one or more flash drives, one or more hard disks, one or more solid state drives, one or more optical drives, etc. In some configurations, the memory 180 can have encoded thereon a computer program for controlling operation of the supplemental computing device 116 (or the computing device 104). In such configurations, the processor 164 can execute at least a portion of the computer program to present content (e.g., user interfaces, images, graphics, tables, reports, and the like), receive content from the computing device 104, transmit information to the computing device 104, and the like.

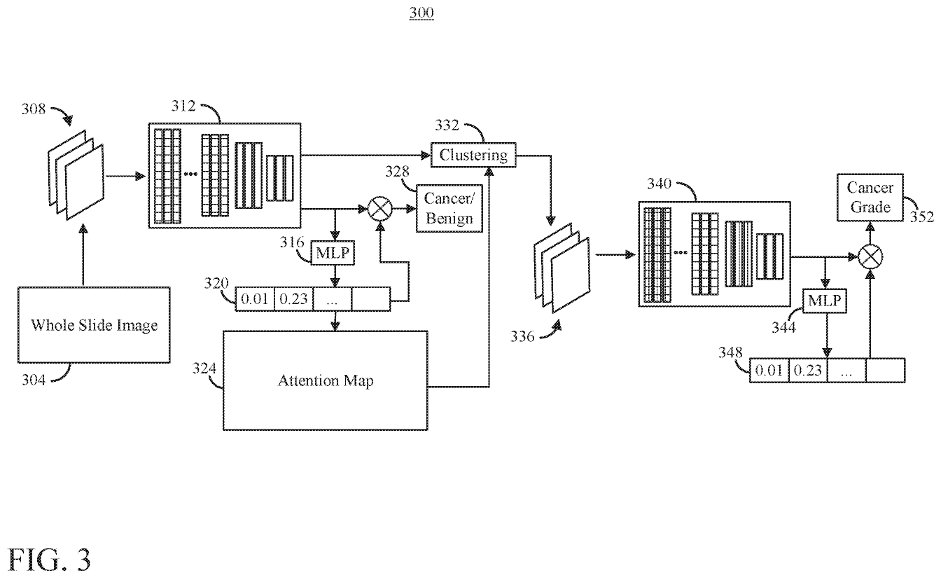

FIG. 3 shows an example of a flow 300 for generating one or more metrics related to the presence of cancer in a patient. More specifically, the flow 300 can generate one or more cancer metrics based on a whole slide image 304 associated with the patient. At least a portion of the flow can be implemented by the image analysis application 132.

The flow 300 can include generating a first number of tiles 308 based on the whole slide image 304. In some configurations, the flow 300 can include generating the first number of tiles 308 by extracting tiles of a predetermined size (e.g., 256×256 pixels) at a predetermined overlap (e.g., 12.5% overlap). The extracted tiles can be taken at a magnification level used in a second number of tiles 336 later in the flow 300. For example, the magnification level of the second number of tiles 336 can be 10× or greater, such as 20×, or 30×, or 40×, or 50× or greater. The flow 300 can include downsampling the extracted tiles to a lower resolution for use with a first trained model 312. In some configurations, the flow 300 can include downsampling the extracted tiles to a 5× magnification level and a corresponding resolution (e.g., 128×128 pixels) to generate the first number of tiles 308. A portion of the original extracted tiles (e.g., the tiles extracted at 10× magnification) can be used as the second number of tiles 336 as described below.

In some configurations, the flow 300 can include preprocessing the whole slide image 304 and/or the first number of tiles 308. Whole slide images may contain many background regions and pen marker artifacts. In some configuration, the flow 300 can include converting the slide at the lowest available magnification into hue, saturation, and value (HSV) color space and thresholding on the hue channel to generate a mask for tissue areas. In some configurations, the flow 300 can include applying morphological operations such as dilation and erosion to fill in small holes and remove isolated points from tissue masks in the whole slide image.

In some configurations, the flow 300 can include selecting the first number of tiles 3087 from the whole slide image 304 using a predetermined image quality metric. In some configurations, the image quality metric can be the blue ratio metric, which may indicative of regions of the whole slide image 304 that have the most nuclei.

The flow 300 can include individually providing each of the tiles 308 to the first trained model 312. In some configurations, the first trained model 312 can include a convolutional neural network (CNN). In some configurations, the first trained model 312 can be trained to generate a number of feature maps based on an input tile. Thus, the first trained model can function as a feature extractor. In some configurations, the convolutional neural network can include a Vggl1 model, such as a Vggl1 model with batch normalization (Vggl1bn). The Vggl1 model, can function as a backbone.

In some configurations, the first trained model 312 can be trained with slide-level annotations in an MIL framework. Specifically, k N×N tiles xᵢ, i∈[1, k] can be extracted from the whole slide image 304, which can contains tens of millions or billions of pixels. The whole slide image can take up Different from supervised computer vision models, in which the label for each tile is provided, only the label for the whole slide image 304 (i.e. the set of tiles) may need to be used, reducing the need for human annotations from a human expert. For example, the label for the whole slide image 304 can be derived from a patient medical file (e.g., what type of cancer the patient had), in contrast to other methods which may require a human expert (e.g., an oncologist) to annotate each tile as indicative of a certain grade of cancer. Each of the tiles can be modeled as instances and the entire slide can be modeled as a bag.

As described above, the first trained model 312 can include a CNN as the backbone to extract instance-level features. An attention module f(·) can be added before a softmax classifier to learn weight distribution α=α₁, α₂, . . . , αₖ for k instances, which indicates importance of k instances for predicting the current bag-level label y (i.e., a slide-level label). The f(·) can be modeled by a multilayer perceptron (MLP). If we denote a set of d dimensional feature vectors from k instances as V∈ [symbol] ^{k×d}, the attention for the ith instance can be defined in Equation 1:

αᵢ=Softmas[U^{T} (tanh Wᵥvᵢ^{T}))] (1)

where U∈ [symbol] ^{h×n} and W∈ [symbol] ^{h×d} are learnable parameters, n is the number of classes, and h is the dimension of the hidden layer. In the first trained model 312, the number of classes n can be two (e.g., benign and cancer). In some configurations, the size of the hidden layer in the attention module h can be 512. Then each tile can have a corresponding attention value learned from the module. Bag-level embedding can be obtained by multiplying learned attentions with instance features.

The flow 300 can include providing the feature maps to a first attention module 316. In some configurations, the first attention module 316 can include a multilayer perceptron (MLP). The first attention module 316 can generate a first number of attention values 320 based on the feature maps generated by the first trained model 312. In some configurations, the first attention module 316 can generate an attention value for a tile based on the feature maps associated with the tile. In some configurations, the flow 300 can include generating an attention map 324 based on the first number of attention values 320. The attention map can include a two-dimensional map of the first number of attention values 320, where each attention value is associated with the same area of the two-dimensional map as the location of the associated tile in the whole slide image 304. The flow 300 can include multiplying the first number of attention values 320 and the feature maps to generate a cancer presence indicator 328, which can indicate whether or not the whole slide image 304 and/or each tile is indicative of cancer or no cancer (i.e., benign).

In some configurations, the first trained model 312 and the first attention module 316 can be included in a first stage model. The first attention module 316 can generate an attention distribution that provides a way to localize informative tiles for the current model prediction. However, the attention-based technique suffers from the same problem as many saliency detection models. Specifically, the model may only focus on the most discriminative input instead of all relevant regions. This problem may not have a large effect on the bag-level classification. Nevertheless, it could affect the integrity of the attention map and therefore affect the performance of the second trained model 340. In some configurations, during training, different instances in the bag can be randomly dropped by setting their pixel values to the mean RGB value of the training dataset; in testing all instances can be used. This method forces the network to discover more relevant instances instead of only relying on the most discriminative ones.

In some configurations, the flow 300 can include selecting informative tiles with attention maps by ranking them by attention values, where the top k percentile are selected. However, this method is highly reliant upon the quality of the learned attention maps, which may not be perfect, especially when there is no explicit supervision. To address this problem, the flow 300 can include selecting tiles based on information from instance feature vectors V. Specifically, instances can be clustered into n clusters based on instance features.

The flow 300 can include clustering 332 the first number of tiles 308. In some configurations, the clustering 332 can include clustering the first number of tiles 308 based on the feature maps and the first number of attention values 320. In some configurations, the flow 300 can include reducing each feature map associated with each tile to a one-dimensional vector. In some configurations, the flow 300 can include reducing feature maps of size 512×4×4 reduced to a 64×4×4 map after a final 1×1 convolution layer, and flattening the 64×4×4 map to form a 1024×1 vector. In some configurations, the flow 300 can include performing principal component analysis (PCA) to reduce the dimension of the 1024×1 instance feature vector to a final instance feature vector, which may have a size of 32×1. The flow 300 can include clustering the final instance feature vectors using K-means clustering in order to group similar tiles. In some configurations, the number of clusters can be set to four.

After the tiles have been clustered, the flow 300 can include determining which tiles to include in the second number of tiles 336. The average attention value for cluster i with m tiles can be computed

$${\overline{a}}_{i} = \frac{1}{m} {\sum }_{i = 1}^{n} {a}_{i}$$

and normalized so that a sums to 1. Clusters with higher average attention are more likely to contain relevant information for slide classification (e.g., given a cancerous slide, clusters containing stroma or benign glands should have lower attention values compared with those containing cancerous regions). The flow 300 can include determining the number of tiles to be selected from each cluster can be determined by the total number of tiles and the average attention of the cluster. For each of the tiles selected from the clusters, the flow 300 can include populating the second number of tiles 336 with tiles corresponding to the same areas of the whole slide image 304 as the tiles selected from the clusters, but having a higher magnification level (e.g., 10×) than used in the first number of tiles 308. For example, the tiles in the second number of tiles 336 can have 256×256 pixels if the first number of tiles 308 have 128×128 pixels and were generated by down sampling tiles at 256×256 pixel resolution.

The second trained model 340 can include at least a portion of the first trained model 312. In some configurations, the number of classes n of the second trained model 340 can be three (e.g., benign, low-grade cancer, and high-grade cancer). In some configurations, low-grade can include Gleason grade 3, and high-grade can include Gleason grade 4 and Gleason grade 5. The flow can include providing each of the second number of tiles 336 to the second trained model 340. The second trained model 340 can output feature maps associated with the second number of tiles 336.

The flow 300 can include providing the feature maps from the second trained model 340 to second attention module 344. In some configurations, the second attention module 344 can include a multilayer perceptron (MLP). The second attention module 344 can generate a second number of attention values 348 based on the feature maps generated by the second trained model 340. In some configurations, the second attention module 344 can generate an attention value for a tile based on the feature maps associated with the tile. The flow 300 can include multiplying the second number of attention values 348 and the feature maps from the second trained model 340 to generate a cancer grade indicator 352, which can indicate whether or not the whole slide image 304 and/or each tile is indicative of no cancer (i.e., benign), low-grade cancer, high-grade cancer, and/or other grades of cancer. In some configurations, the second trained model 340 and the second attention module 344 can be included in a second stage model.

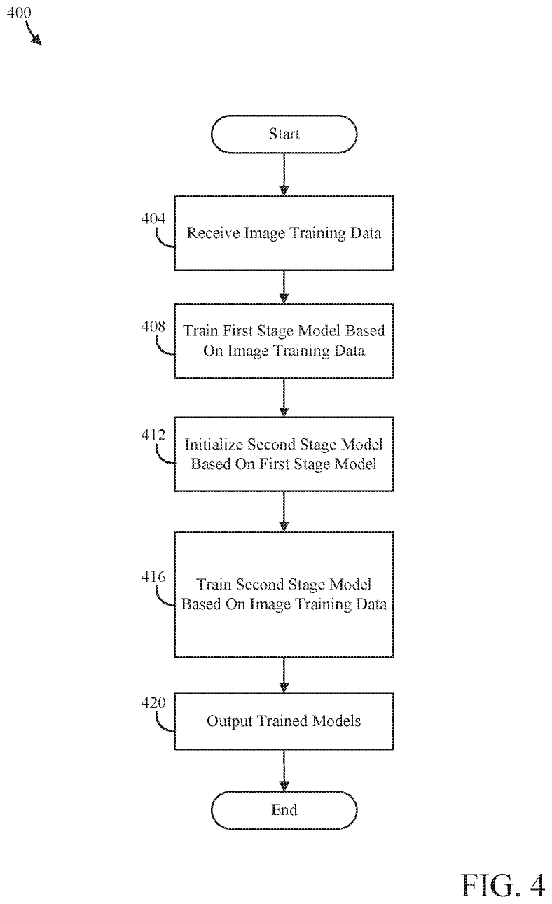

Referring to FIG. 3 as well as FIG. 4, an exemplary process 400 for training a first stage model and a second stage model is shown. The process 400 can be included in the sample image analysis application 132.

At 404, the process 400 can receive image training data. In some configurations, the image training data can include a number of whole slide images annotated with a presence of cancer and/or a cancer grade for the whole slide image. For example, each whole slide image can be annotated as benign, low-grade cancer, or high-grade cancer. In some configurations, low-grade cancer and high-grade cancer annotations can be normalized to “cancer” for training the first model 312. In some configurations, low-grade can include Gleason grade 3, and high-grade can include Gleason grade 4 and Gleason grade 5. The process 400 can include preprocessing the whole slide images. In some configurations, the process 400 can include converting each WSI at the lowest available magnification into HSV color space and thresholding on the hue channel to generate a mask for tissue areas. In some configurations, the process 400 can include performing morphological operations such as dilation and erosion to the whole slide images in order to fill in small holes and remove isolated points from tissue masks. In some configurations, after optional preprocessing, the process 400 can include generating a number set of tiles for the slides. Each tile can be of size 256×256 pixels at 10× was extracted from the grid with 12.5% overlap. In some configurations, the tiles extracted at 10× can be included in a second model training set. The process 400 may remove tiles that contain less than 80% tissue regions. The number of tiles generated per slide may range from about 100 to about 300. In some configurations, the process 400 can include downsampling the number set of tiles to 5× to generate a first model training set. In some configurations, the image training data can include the first model training set and the second model training set, with any generating preprocessing, filtering, etc. of the tiles pre-performed. In some configurations, the training data cab include a tile-level dataset including a number of slides annotated at the pixel-level (i.e., each pixel is labeled as benign, low-grade, and high grade).

At 408, the process 400 can train a first stage model based on the training data. The first stage model can include a first extractor and the first attention module 316. Once trained, the first extractor can be used as the first trained model 312. In some configurations, a Vggl1 model such as a Vggl1bn model can be used as the first extractor. In some configurations, the Vggl1bn can be initialized with weights pretrained on ImageNet.

In some configurations, the first extractor can be trained based on a tile-level dataset. In some configurations, the tile-level dataset can include a number of slides annotated at the pixel-level (i.e., each pixel is labeled as benign, low-grade, and high grade). The low-grade and high-grade classifications can be normalized to “cancer” for the first extractor. The slides can be annotated by a human expert, such as a pathologist. For example, a pathologist can circle and grade the major foci of a tumor in a slide and/or tile as either low-grade, high-grade, or benign areas. The number of annotated slides needed to generate the tiles in the tile-level dataset may be relatively low as compared to a number of slide-level annotated slides used to train other aspects of the first stage model, as will be discussed below. For example, only about seventy slides may be required to generate the tile-level dataset, while the slide-level dataset may include thousands of slide-level annotated slides. In some configurations, the process 400 can randomly select tiles from the tile-level dataset to train the first extractor. The tiles in the tile-level dataset can be taken at 10×, and downsampled to 5× as described above in order to train the first extractor. In some configurations, the process 400 can train the first extractor using the randomly selected tiles using a batch size of fifty and an initial learning rate of 1e⁻⁵. After training the first extractor, the fully connected layers can be replaced by a 1×1 convolutional layer to reduce the feature map dimension, outputs of which can be flattened and used as instance feature vectors V in the MIL model for slide classification.

After the first extractor is trained on the randomly selected tiles, the process 400 can fix the feature extractor and train the first attention module 316 and associated classification layer were trained with a predetermine learning rate, such as 1e⁻⁴, for a predetermined number of epochs, such as ten epochs. The process 400 can then train the last two convolutional blocks for the Vggl1bn model with a learning rate of 1e−5 for the feature extractor, and a learning rate of 1e⁻⁴ for the classifier for 90 epochs. The process 400 can reduce learning rates by a factor of 0.1 if the validation loss did not decrease for the last 10 epochs. In some configurations, the process 400 can drop instances (e.g., randomly drop) at a predetermined instance dropout rate (e.g., 0.5).

In some configurations, after training the first attention module 316 and the associated classification layer, the process 400 can concurrently train the last two convolutional blocks for the Vggl1bn model with a learning rate of 1e⁻⁵ and the classifier with a learning rate of 1e⁻⁴ for the classifier, for a predetermined number of epochs (e.g. about ninety epochs). The process 400 can reduce learning rates by a factor of 0.1 if the validation loss does not decrease for ten consecutive epochs. In some configurations, process 400 can reduce feature maps of size 512×4×4 to 64×4×4 after the 1×1 convolution, and then flattened to form a 1024×1 vector using a fully connected layer embedded it into a 1024×1 instance feature vector.

At 412, the process 400 can initialize the second stage model based on the first stage model. More specifically, the process can initialize a second extractor included in the second stage model with the weights of the first extractor. The second extractor can include at least a portion of the first extractor. For example, the second extractor can include a Vggl1bn model.

At 416, the process 400 can train the second stage model based on the image training data. In some configurations, the process 400 can determine which tiles in the number set of tiles can be in the second model training set in order to train the second stage model by clustering outputs from the first stage model. For example, the process 400 can cluster the outputs and select the tiles as described above in conjunction with the flow 300 (e.g., at the clustering 332). The selected tiles can then be provided to the second stage model at the magnification associated with the second stage model (e.g., 10×). The process 400 can train the second stage model with the second feature extractor fixed. The process 400 can train the second attention module 344 for five epochs with the same hyperparameters (e.g., learning rates, reduction of learning rates, etc.) as the first attention module 316. Once trained, the second feature extractor can be used as the second trained model 340.

At 420, the process 400 can output the trained first stage mode and the trained second stage model. More specifically, the process 400 can output the first trained model 312, the first attention model 316, the second trained model 340, and the second attention module 344. The first trained model 312, the first attention model 316, the second trained model 340, and the second attention module 344 can then be implemented in the flow 300. In some configurations, the process 400 can cause the first trained model 312, the first attention model 316, the second trained model 340, and the second attention module 344 to be saved to a memory, such as the memory 160 and/or the memory 180 in FIG. 2.

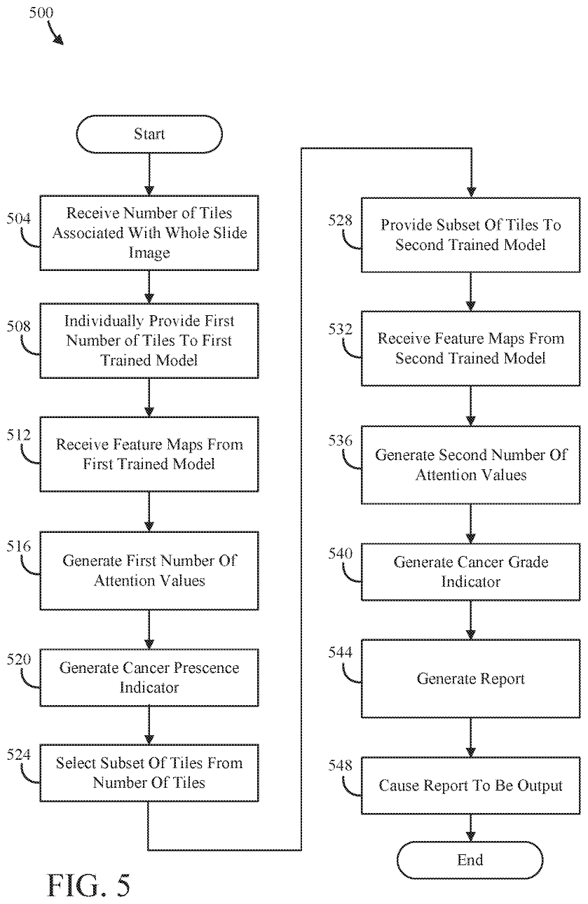

Referring to FIG. 3 as well as FIG. 5, an exemplary process 500 for generating cancer predictions for a patient is shown. The process 500 can be included in the sample image analysis application 132.

At 504, the process 500 can receive number of tiles associated with a whole slide image. The whole slide image can be associated with a patient. In some configurations, the whole slide image can be the whole slide image 304 in FIG. 3. In some configurations, the number of tiles can include a first number of tiles taken at a first magnification level (e.g., 5×) from a whole slide image, and a second number of tiles taken at a second magnification level (e.g., 10× or greater) from the whole slide image. In some configurations, the first number of tiles can include the first number of tiles 308 in FIG. 3. In some configurations, the second number of tiles can include the second number of tiles 336 in FIG. 3. Each of the first number of tiles can be associated with a tile included in the second number of tiles.

At 508, the process 500 can individually provide each of the first number of tiles to a first trained model. In some configurations, the first trained model can be the first trained model 312 in FIG. 3.

At 512, the process 500 can receive feature maps associated with the first number of tiles from the first trained model.

At 516, the process 500 can generate a first number of attention values based on the feature maps associated with the first number of tiles. In some configurations, the process 500 can provide each of the feature maps to a first attention model. In some configurations, the first attention model can be the first attention model 316 in FIG. 3. The process 500 can receive a first number of attention values from the first attention model. Each attention value can be associated with each tile included in the first number of tiles.

At 520, the process 500 can generate a cancer presence indicator. In some configurations, the process 500 can multiply the first number of attention values and the feature maps to generate a cancer presence indicator as described above. In some configurations, the cancer presence indicator can be the cancer presence indicator 328 in FIG. 3.

At 524, the process 500 can select a subset of tiles from the number of tiles. In some configurations, the process 500 can include clustering the first number of tiles based on the feature maps and the first number of attention values. In some configurations, the process 500 can include reducing each feature map associated with each tile to a one-dimensional vector. In some configurations, the process 500 can include reducing feature maps of size 512×4×4 reduced to a 64×4×4 map after a final 1×1 convolution layer, and flattening the 64×4×4 map to form a 1024×1 vector. In some configurations, the process 500 can include performing PCA to reduce the dimension of the 1024×1 instance feature vector to a final instance feature vector, which may have a size of 32×1. The process 500 can include clustering the final instance feature vectors using K-means clustering in order to group similar tiles. In some configurations, the number of clusters can be set to four. The subset of tiles to be used in further processing can be selected based on the number of tiles and the average attention value per cluster as described above.

At 528, the process 500 can provide the subset of tiles to a second trained model. In this way, the subset of tiles can function as the second number of tiles 336 in FIG. 3. In some configurations, the second trained model can be the second trained model 340 in FIG. 3.

At 532, the process 500 can receive feature maps associated with the subset of tiles from the second trained model.

At 536, the process 500 can generate a second number of attention values based on the feature maps associated with the subset of tiles. In some configurations, the process 500 can provide each of the feature maps to a second attention model. In some configurations, the first attention model can be the second attention model 344 in FIG. 3. The process 500 can receive a second number of attention values from the second attention model. Each attention value can be associated with each tile included in the subset of tiles.

At 540, the process 500 can generate a cancer grade indicator. In some configurations, the process 500 can include multiplying the second number of attention values and the feature maps from the second trained model to generate the cancer grade indicator, which can indicate whether or not the whole slide image 304 and/or each tile is indicative of no cancer (i.e., benign), low-grade cancer, high-grade cancer, and/or other grades of cancer.

At 544, the process 500 can generate a report. The report can be associated with the patient. In some configurations, the process 500 can generate the report based on the cancer presence indicator, the cancer grade indicator, the first number of attention values, the second number of attention values, and/or the whole slide image.

At 548, the process 500 can cause the report to be output to at least one of a memory or a display. In some configurations, at 548, the process 500 can cause the report to be displayed on a display (e.g., the display 108, the display 148 in the computing device 104, and/or the display 168 in the supplemental computing device 116). In some configurations, at 548, the process 500 can cause the report to be saved to memory (e.g., the memory 160, in the computing device 104 and/or the memory 180 in the supplemental computing device 116).

### Experiment

An experiment to test the performance of the techniques presented above is now described. Cedars Sinai dataset. CNN feature extractors for both stages were pre-trained with a relatively small dataset with manually drawn ROIs from the Department of Pathology at Cedars-Sinai Medical Center (IRB approval numbers: Pro00029960 and Pro00048462). The dataset contains two parts. 1) 513 tiles of size 1200×1200 extracted from prostatectornies of 40 patients, which contain low-grade pattern (Gleason grade 3), high-grade pattern (Gleason grade 4 and 5), benign (BN), and stromal areas. These tiles were annotated by pathologists at the pixel-level. 2) 30 WSIs from prostatectornies of 30 patients. These slides were annotated by a pathologist who circled and graded the major foci of tumor as either low-grade, high-grade, or BN areas.

The scanning objective for all slides and tiles was set at 20×(0.5 μm per pixel). To use this dataset for tile classification, 11,595 tiles of size 256×256 at were randomly sampled at 10× from annotated regions. This dataset will be referred to as the tile-level dataset in the following sections.

UCLA dataset: The MIL model is further trained with a large-scale dataset with only slide-level annotations. The dataset contains prostate biopsy slides from the Department of Pathology and Laboratory Medicine at the University of California, Los Angeles (UCLA). A balanced number of low-grade, high-grade, and benign cases were randomly sampled, resulting in 3,521 slides from 718 patients. The dataset was randomly divided based on patients for model training, validation, and testing to ensure the same patient would not be included in both training and testing. Labels for these slides were retrieved from pathology reports. For simplicity, this dataset is referred to as the slide-level dataset in the following sections.

Data preprocessing: Since WSIs may contain a lot of background regions and pen marker artifacts, some configurations of the model include converting the slide at the lowest available magnification into HSV color space and thresholding on the hue channel to generate a mask for tissue areas. Morphological operations such as dilation and erosion were applied to fill in small holes and remove isolated points from tissue masks. Then, a set of instances (i.e. tiles) for one bag (i.e. slide) of size 256×256 at 10× was extracted from the grid with 12.5% overlap. Tiles that contained less than 80% tissue regions were removed from analysis. The number of tiles in the majority of slides ranged from 100 to 300. The same color normalization algorithm was performed on tiles from both UCLA and Cedars Sinai datasets. Tiles at 10× were downsampled to 5x for the first stage of model training. Blue ratio selection: A blue ratio image may be used to select relevant regions in the WSI. The blue ratio image as defined in Equation 2 below reflects the concentration of the blue color, so it can detect regions with the most nuclei.

$$BR = \frac{100 \times B}{1 + R + G} \times \frac{256}{1 + R + G + B} \qquad ( 2 )$$

In equation 2, R, G, B are the red, green and blue channels in the whole slide image 304, respectively. The top k percentile of tiles with highest blue ratio can then be selected. In some configurations, this method, br-two-stage, is used as the baseline for ROI detection.

CNN feature extractor: In some configurations, a Vggl1 model with batch normalization (Vggl1bn) is used as the backbone for the feature extractor in both 5× and 10× models. The Vggl1bn may be initialized with weights pretrained on ImageNet. The feature extractor was first trained on the tile-level dataset for tile classification. After that, the fully connected layers were replaced by a 1×1 convolutional layer to reduce the feature map dimension, outputs of which were flattened and used as instance feature vectors V in the MIL model for slide classification. The batch size of the tile-level model was set to 50, the initial learning rate was set to 1e⁻⁵. Two-stage classification model

The first stage model was developed for cancer versus non-cancer classification. The knowledge from the tile-level dataset was transferred by initializing the feature extractor with learned weights. The feature extractor was initially fixed, while the attention module and classification layer were trained with a learning rate at 1e⁻⁴ for 10 epochs. Then, the last two convolutional blocks for the Vggl1bn model were fine-tuned with a learning rate of 1e⁻⁵ for the feature extractor, and a learning rate of 1e⁻⁴ for the classifier for 90 epochs. Learning rates were reduced by 0.1 if the validation loss did not decrease for the last 10 epochs. The instance dropout rate was set to 0.5. Feature maps of size 512×4×4 were reduced to 64×4×4 after the 1×1 convolution, and then flattened to form a 1024×1 vector. A fully connected layer embedded it into a 1024×1 instance feature vector. The size of the hidden layer in the attention module h was set to 512. The model with the highest accuracy on the validation set was utilized to generate attention maps. PCA was used to reduce the dimension of the instance feature vector to 32. K-means clustering was then performed to group similar tiles. The number of clusters was set to 4. Hyper-parameters were tuned on the validation set. Selected tiles at 10× were fed into the second-stage grading model. Similarly, the feature extractor was initialized with weights learned from the tile-level classification. The model was trained for five epochs with the feature extractor fixed. Other hyperparameters were the same as the first-stage model. Both tile- and slide-classification models were implemented in PyTorch 0.4, and trained using one NVIDIA Titan X GPU.

### Results

The performance of most state-of-the-art models for prostate WSIs classification is summarized in Table 1.

TABLE 1

| Models | Accuracy (%) | Dataset | Classification Task |
|---|---|---|---|
| Zhou et al. | 75.00 | 368 slides | G3 + G4 and |
| | | | G4 + G3 slides |
| Xu et al. | 79.00 | 312 slides | GS 6, GS 7, and |
| | | | GS 8 slides |
| Nagpal et al. | 70.00 | 112 million | 4 Gleason groups |
| | | patches and | |
| | | 1490 slides | |
| Model in | 85.11 | 3521 slides | Benign, low-grade, |
| Accordance with | | | high-grade slides |
| Flow 300 | | | |

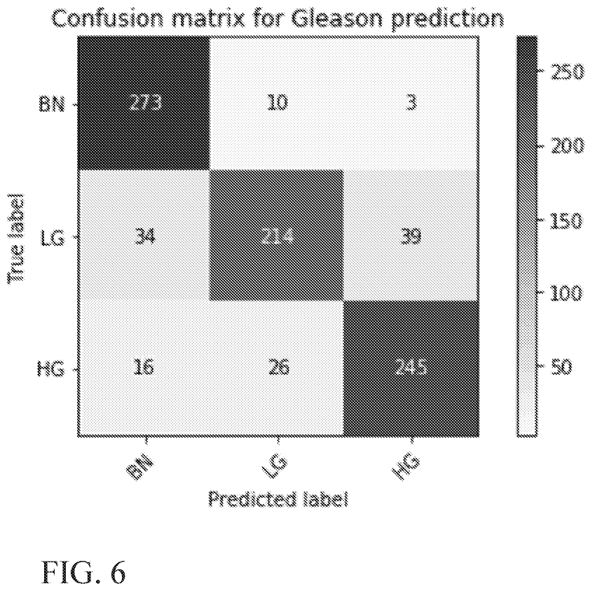

FIG. 6 shows a Confusion matrix for Gleason grade classification on the test set. As shown in Table 1, the task of Zhou et al.'s work is the closet to the presented study, with the main difference being that the model in accordance with the flow 300 included a benign class. The work by Xu et al. can be considered relatively easy compared with the task of classifying between benign, low-grade, and high-grade, since differentiating G3+G4 versus G3+G4 is non-trivial and often has the largest inter-observer variability. The model developed by Nagpal et al. achieved a lower accuracy compared with the model in accordance with the flow 300 in FIG. 3. However, their model predicted more classes, but relied on tile-level labels, which may not be directly comparable.

Several experiments were performed to evaluate the effects of different components on model performance. Specifically, in experiment att-two-stage, informative tiles were selected based only on attention maps generated from the first stage model, while in the att-cluster-two-stage model, both instance features and attention maps were used as discussed above. The br-two-stage model was implemented to evaluate the effectiveness of the attention-based ROI detection. To investigate the instance dropout, another model was trained without instance dropout, att-no-dropout. To evaluate the contribution of knowledge transferred from the Cedars dataset, a model was trained without transfer learning. For simplicity, this model is denoted as no-transfer. The one-stage model was trained with tiles only from 5×.

TABLE 2

| Models | Accuracy (%) |
|---|---|
| one-stage | 77.80 |
| hr-two-stage | 80.11 |
| att-two-stage | 81.86 |
| att-no-dropout | 79.65 |
| no-transfer | 84.30 |
| att-cluster-two-stage | 85.11 |

Table 2 shows that that the model with clustering-based attention achieved the best performance with the average accuracy over 7% higher than the one-stage model, over 5% higher than the vanilla attention model (i.e. att-no-dropout). All two-stage models outperformed the one-stage, which utilized all tiles at 5× to predict cancer grading. This is likely due to the fact that important visual features, such as those from nuclei, may only be available at higher resolution. As discussed above, attention maps learned in the weakly-supervised model are likely to be only focused on the most discriminative regions instead of the whole part, which could potentially harm model performance.

In testing, clustering with instance features reduced false positive tiles. Pen markers, which may indicate potential suspicious areas, were drawn by pathologists during the diagnosis. This information was not used-for model training, since it was not always available. In testing, instance dropout was shown to improve performance as compared to models without instance dropout. The attention map trained without instance dropout failed to identify the entire region of interest.

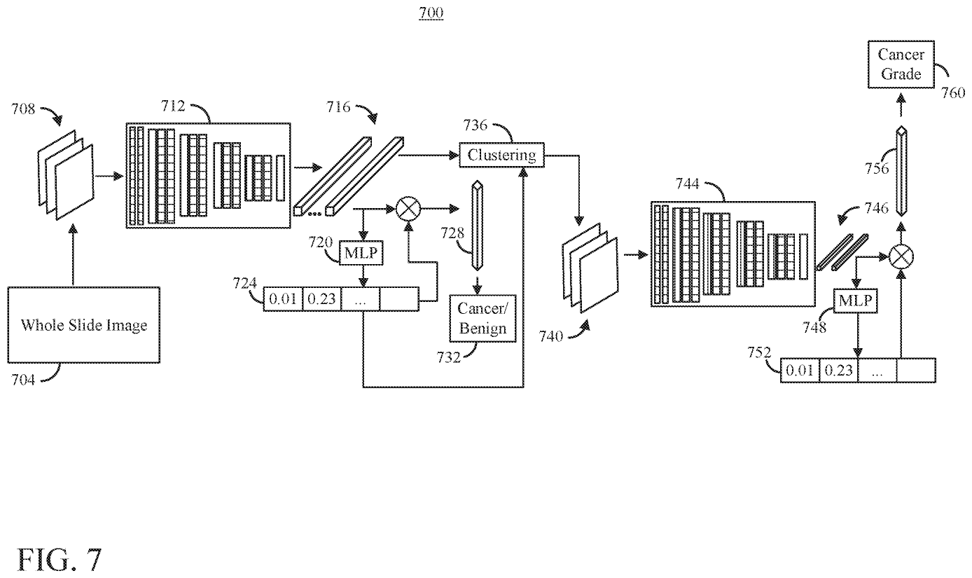

Another exemplary flow for generating cancer indicators is now discussed. FIG. 7 shows an example of a flow 700 for generating one or more metrics related to the presence of cancer in a patient. More specifically, the flow 700 can generate one or more cancer metrics based on a whole slide image 704 associated with the patient. At least a portion of the flow can be implemented by the image analysis application 132.

The flow 700 can include generating a first number of tiles 708 based on the whole slide image 704. In some configurations, the flow 700 can include generating the first number of tiles 708 by extracting tiles of a predetermined size (e.g., 256×256 pixels) at a predetermined overlap (e.g., 12.5% overlap). The extracted tiles can be taken at a magnification level used in a second number of tiles 740 later in the flow 700. For example, the magnification level of the second number of tiles 740 can be 10× or greater, such as 20×, or 30×, or 40×, or 50× or greater.

The flow 700 can include downsampling the extracted tiles to a lower resolution for use with a first trained model 712. In some configurations, the flow 700 can include downsampling the extracted tiles to a 5× magnification level and a corresponding resolution (e.g., 128×128 pixels) to generate the first number of tiles 708. A portion of the original extracted tiles (e.g., the tiles extracted at 10× magnification) can be used as the second number of tiles 740 as described below.

In some configurations, the flow 700 can include preprocessing the whole slide image 704 and/or the first number of tiles 708. Whole slide images may contain many background regions and pen marker artifacts. In some configuration, the flow 700 can include converting the slide at the lowest available magnification into HSV color space and thresholding on the hue channel to generate a mask for tissue areas. In some configurations, the flow 700 can include applying morphological operations such as dilation and erosion to fill in small holes and remove isolated points from tissue masks in the whole slide image.

In some configurations, the flow 700 can include selecting the first number of tiles 7087 from the whole slide image 704 using a predetermined image quality metric. In some configurations, the image quality metric can be the blue ratio metric, which may indicative of regions of the whole slide image 704 that have the most nuclei.

The flow 700 can include individually providing each of the tiles 708 to the first trained model 712. In some configurations, the first trained model 712 can include a CNN. In some configurations, the first trained model 712 can be trained to generate a number of feature vectors based on an input tile. Thus, the first trained model can function as a feature extractor. In some configurations, the convolutional neural network can include a Vggl1 model, such as a Vggl1 model with batch normalization (Vggl1bn). The Vggl1 model can function as a backbone. In some configurations, the first trained model 712 can include a 1×1 convolutional layer added after the last convolutional layer of the VGGl1bn model. The 1×1 convolutional layer can reduce dimensionality and generate k×256×4×4 instance-level feature maps for k tiles. The flow 700 can include flattening the feature maps and feeding the feature maps into a fully connected layer with 256 nodes, followed by ReLU and dropout layers (in training only), which can output the first number of feature vectors 716.

The first number of feature vectors 716 can be a k×256 instance embedding matrix, which was forwarded into the first attention module 720. In some configurations, the first attention module 720, which can generate a k×n attention matrix for n prediction classes, can include two fully connected layers with dropout, tanh non-linear activations, and a softmax layer. In some configurations, the flow 700 can include multiplying instance embeddings with attention weights, producing a n×256 bag-level representation, which can be flattened and input into the final classifier. The probability of instance dropout can be set to 0.5 during training.

In some configurations, the first trained model 712 can be trained with slide-level annotations in an MIL framework. Specifically, k N×N tiles xᵢ, i∈[1, k] can be extracted from the whole slide image 704, which can contains gigabytes of pixels. Each tile can have different instance-level labels yᵢ, i∈[1, k]. During training, only the label for a set of instances (i.e., bag-level) Y may be required. Based on the MIL assumption, a positive bag should contain at least one positive instance, while a negative bag contains all negative instances in a binary classification scenario as defined in Equation 3 below. The flow 700 can include a first attention module 720 that aggregates instance features and forms the bag-level representation, instead of using a pre-defined function, such as maximum or mean pooling.

$$Y = \{ \begin{matrix}0 & if f ∀ i ∈ [ 1 , k ] , {y}_{i} = 0 \\ 1 & otherwise\end{matrix} \qquad ( 3 )$$

The first trained model 712 can include a CNN. The CNN can transform each instance into a d dimensional feature vector vᵢ∈ [symbol] ^{d}. The feature vector may be referred to as a tile-level feature vectors. The first trained model 712 can output a first number of feature vectors 716 based on the first number of tiles 708. A permutation invariant function f(·) can be applied to aggregate and project k instance-level feature vectors into a joint bag-level representation. In some configurations, the flow 700 can include providing the first number of feature vectors 716 to a first attention module 720, which can be a multilayer perceptron-based attention module. In some configurations, the first attention module 720 can be modeled as f(·), which produces a combined bag-level feature vector v′ and a set of attention values representing the relative contribution of each instance as defined in Equation (4):

v′=f(V)=Σᵢ₌₁^{k}αᵢvᵢ

α=Softmax[u^{T} tanh(WV^{T})] (4)

where V∈ [symbol] ^{k×d} contains the feature vectors for k tiles, u∈ [symbol] ^{h×1} and W∈ [symbol] ^{h×d} are parameters in the first attention module 720, and h denotes the dimension of the hidden layer. The slide-level prediction can be obtained by applying a fully connected layer to the bag-level representations v′. Both the first trained model 712 and the first attention module 720 can be differentiable, and can be trained end-to-end using gradient descent. The first attention module 720 can provide a more flexible way to incorporate information from instances while also localizing informative tiles.

This framework encounters similar problems as other saliency detection models. In particular, instead of detecting the all informative regions, the learned attention map can be highly sparse with very few positive instances having large values. This issue may be caused by the underlying MIL assumption that only one positive instance needs to be detected for a bag to be classified as positive. While the bag-level prediction may not be significantly influenced by this problem, it can affect the performance of our classification stage model, which relies on informative tiles selected by the learned attention map. In some configurations, to encourage the first trained model 712 and/or the first attention module 720 to select more relevant tiles, an instance dropout technique can be used during training. Specifically, training can include randomly dropping instances during training, while all instances are used during model evaluation. In some configurations, to ensure the distribution of inputs for each node in the network remains the same during training and testing, the flow 700 can include setting pixel values of dropped instances to be the mean RGB value of the dataset. This form of instance dropout can be considered a regularization method that prevents the network from relying on only a few instances for bag-level classification.

Different from supervised computer vision models, in which the label for each tile is provided, only the label for the whole slide image 704 (i.e. the set of tiles) may need to be used, reducing the need for human annotations from a human expert. For example, the label for the whole slide image 704 can be derived from a patient medical file (e.g., what type of cancer the patient had), in contrast to other methods which may require a human expert (e.g., an oncologist) to annotate each tile as indicative of a certain grade of cancer. Each of the tiles can be modeled as instances and the entire slide can be modeled as a bag.

An intuitive approach to localize suspicious regions with learned attention maps is to use the top q percent of tiles with the highest attention weights. However, the percentage of cancerous regions can vary across different cases. Therefore, using a fixed q may cause over selection for slides with small suspicious regions and under selection for those with large suspicious regions. Moreover, the flow 700 can use an attention map, which can be learned without explicit supervision at the pixel- or region-level.

To address these challenges, we incorporate information embedded in instance-level representations by selecting informative tiles from clusters. Specifically, instance representations obtained from the MIL model are projected to a compact latent embedding space using PCA as described above.

The flow 700 can include providing the first number of feature vectors 716 to the first attention module 720. In some configurations, the first attention module 720 can include a multilayer perceptron (MLP). The first attention module 720 can generate a first number of attention values 724 based on the first number of feature vectors 716 generated by the first trained model 712. In some configurations, the first attention module 720 can generate an attention value for a tile based on the feature vectors associated with the tile. The flow 700 can include aggregating instance-level representations into a bag-level feature vector 728 and producing a saliency map that represents relative importance of each tile for predicting slide-level labels. The flow 700 can include applying a fully connected layer to the bag-level feature vector 728 in order to generate a cancer presence indicator 732. The cancer presence indicator 732 can indicate whether or not the whole slide image 704 is indicative of cancer or no cancer (i.e., benign).

In some configurations, the first trained model 712 and the first attention module 720 can be included in a first stage model. The first attention module 720 can generate an attention distribution that provides a way to localize informative tiles for the current model prediction. However, the attention-based technique suffers from the same problem as many saliency detection models. Specifically, the model may only focus on the most discriminative input instead of all relevant regions. This problem may not have a large effect on the bag-level classification. Nevertheless, it could affect the integrity of the attention map and therefore affect the performance of the second trained model 744. In some configurations, during training, different instances in the bag can be randomly dropped by setting their pixel values to the mean RGB value of the training dataset; in testing all instances can be used. This method forces the network to discover more relevant instances instead of only relying on the most discriminative ones.

In some configurations, the flow 700 can include selecting informative tiles with attention maps by ranking them by attention values, where the top k percentile are selected. However, this method is highly reliant upon the quality of the learned attention maps, which may not be perfect, especially when there is no explicit supervision. To address this problem, the flow 700 can include selecting tiles based on information from instance feature vectors V. Specifically, instances can be clustered into n clusters based on instance features.

The flow 700 can include clustering 736 the first number of tiles 708. In some configurations, the clustering 736 can include clustering the first number of tiles 708 based on the feature vectors 716 and the first number of attention values 724. In some configurations, the flow 700 can include reducing each feature map associated with each tile to a one-dimensional vector. In some configurations, the flow 700 can include reducing feature vectors using PCA to reduce the dimension of the feature vectors. The flow 700 can include clustering the final instance feature vectors (i.e., the vectors reduced using PCA) using K-means clustering in order to group similar tiles. In some configurations, the number of clusters can be set to four.

After the tiles have been clustered, the flow 700 can include determining which tiles to include in the second number of tiles 740. The average attention value for cluster i with m tiles can be computed

$${\overline{a}}_{i} = \frac{1}{m} {\sum }_{i = 1}^{n} {a}_{i}$$

and normalized so that α̅ sums to 1. Clusters with higher average attention are more likely to contain relevant information for slide classification (e.g., given a cancerous slide, clusters containing stroma or benign glands should have lower attention values compared with those containing cancerous regions). The flow 700 can include determining the number of tiles to be selected from each cluster can be determined by the total number of tiles and the average attention of the cluster. For each of the tiles selected from the clusters, the flow 700 can include populating the second number of tiles 740 with tiles corresponding to the same areas of the whole slide image 704 as the tiles selected from the clusters, but having a higher magnification level (e.g., 10×) than used in the first number of tiles 708. For example, the tiles in the second number of tiles 740 can have 256×256 pixels if the first number of tiles 708 have 128×128 pixels and were generated by down sampling tiles at 256×256 pixel resolution.

The second trained model 744 can include at least a portion of the first trained model 712. In some configurations, the number of classes n of the second trained model 744 can be three (e.g., benign, low-grade cancer, and high-grade cancer). In some configurations, low-grade can include Gleason grade 3, and high-grade can include Gleason grade 4 and Gleason grade 5. The flow can include providing each of the second number of tiles 740 to the second trained model 744. The second trained model 744 can output feature vectors 746 associated with the second number of tiles 740.

The flow 700 can include providing the feature vectors 746 from the second trained model 744 to second attention module 748. In some configurations, the second attention module 748 can include a MLP. The second attention module 748 can generate a second number of attention values 752 based on the feature vectors 746 generated by the second trained model 744. In some configurations, the second attention module 748 can generate an attention value for a tile based on the feature vectors 746 associated with the tile. The flow 700 can include aggregating instance-level representations from the second trained model 744 into a second bag-level feature vector 756 and producing a saliency map that represents relative importance of each tile for predicting slide-level labels. The flow 700 can include applying a fully connected layer to the bag-level feature vector 728 in order to generate a cancer grade indicator 760, which can indicate whether or not the whole slide image 704 and/or each tile is indicative of no cancer (i.e., benign), low-grade cancer, high-grade cancer, and/or other grades of cancer. In some configurations, the second trained model 744 and the second attention module 748 can be included in a second stage model.

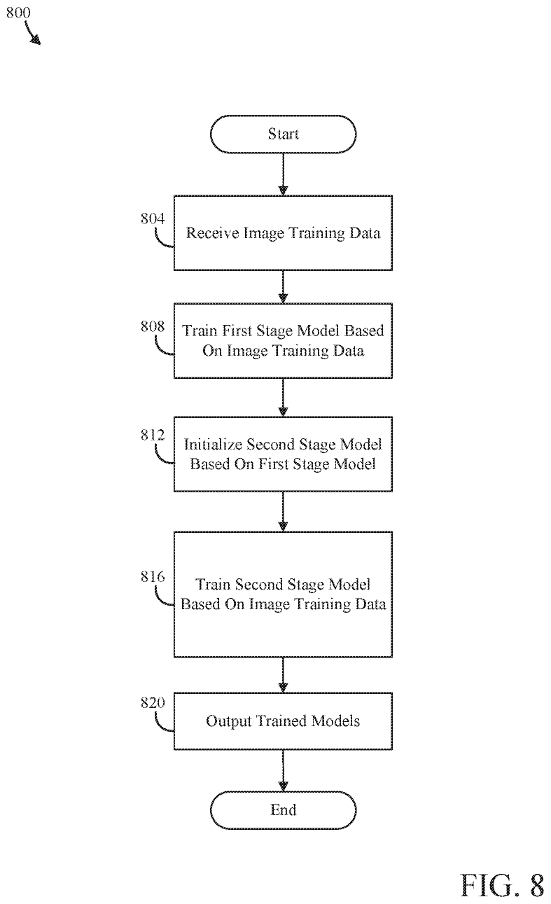

Referring to FIG. 7 as well as FIG. 8, an exemplary process 800 for training a first stage model and a second stage model is shown. The process 800 can be included in the sample image analysis application 132.

At 804, the process 800 can receive image training data. In some configurations, the image training data can include a number of whole slide images annotated with a presence of cancer and/or a cancer grade for the whole slide image. For example, each whole slide image can be annotated as benign, low-grade cancer, or high-grade cancer. In some configurations, low-grade cancer and high-grade cancer annotations can be normalized to “cancer” for training the first model 312. In some configurations, low-grade can include Gleason grade 3, and high-grade can include Gleason grade 8 and Gleason grade 5. The process 800 can include preprocessing the whole slide images. In some configurations, the process 800 can include converting each WSI at the lowest available magnification into HSV color space and thresholding on the hue channel to generate a mask for tissue areas. In some configurations, the process 800 can include performing morphological operations such as dilation and erosion to the whole slide images in order to fill in small holes and remove isolated points from tissue masks. In some configurations, after optional preprocessing, the process 800 can include generating a number set of tiles for the slides. Each tile can be of size 256×256 pixels at 10× was extracted from the grid with 12.5% overlap. In some configurations, the tiles extracted at 10× can be included in a second model training set. The process 800 may remove tiles that contain less than 80% tissue regions. The number of tiles generated per slide may range from about 100 to about 300. In some configurations, the process 800 can include downsampling the number set of tiles to 5× to generate a first model training set. In some configurations, the image training data can include the first model training set and the second model training set, with any generating preprocessing, filtering, etc. of the tiles pre-performed. In some configurations, the training data cab include a tile-level dataset including a number of slides annotated at the pixel-level (i.e., each pixel is labeled as benign, low-grade, and high grade).

At 808, the process 800 can train a first stage model based on the training data. The first stage model can include a first extractor and the first attention module 724. Once trained, the first extractor can be used as the first trained model 712. In some configurations, a Vggl1 model such as a Vggl1bn model can be used as the first extractor. In some configurations, the Vggl1bn can be initialized with weights pretrained on ImageNet. In some configurations, the process 800 can train the first attention module 724 and the classifier with the first extractor frozen for three epochs. The process 800 can the train the last three VGG blocks in the first extractor together with the first attention module 724 and classifier for ninety-seven epochs. In some configurations, the initial learning rates for the feature extractor can be set at1×10⁻⁵ and 5×10⁻⁵ for the first attention module 724 and the classifier, respectively. In some configurations, the learning rate can be decreased by a factor of 10 if the validation loss did not improve for the last 10 epochs. In some configurations, the process 800 can include training the first stage model using an Adam optimizer and a batch size of one.

At 812, the process 800 can initialize the second stage model based on the first stage model. More specifically, the process can initialize a second extractor included in the second stage model with the weights of the first extractor. The second extractor can include at least a portion of the first extractor. For example, the second extractor can include a Vggl1bn model.

At 816, the process 800 can train a second stage model based on the training data. The second stage model can include a second extractor and the second attention module 748. Once trained, the second extractor can be used as the second trained model 744. In some configurations, a Vggl1 model such as a Vggl1bn model can be used as the second extractor. In some configurations, the Vggl1bn can be initialized with weights pretrained on ImageNet. In some configurations, the process 800 can train the second attention module 748 and the classifier with the second extractor frozen for three epochs. The process 800 can the train the last three VGG blocks in the second extractor together with the second attention module 748 and classifier for ninety-seven epochs. In some configurations, the initial learning rates for the feature extractor can be set at 1×10⁻⁵ and 5×10⁻⁵ for the second attention module 748 and the classifier, respectively. In some configurations, the learning rate can be decreased by a factor of 10 if the validation loss did not improve for the last 10 epochs. In some configurations, the process 800 can include training the second stage model using an Adam optimizer and a batch size of one.

At 820, the process 800 can output the trained first stage mode and the trained second stage model. More specifically, the process 800 can output the first trained model 712, the first attention model 720, the second trained model 744, and the second attention module 748. The first trained model 712, the first attention model 720, the second trained model 744, and the second attention module 748 can then be implemented in the flow 700. In some configurations, the process 800 can cause the first trained model 712, the first attention model 720, the second trained model 744, and the second attention module 748 to be saved to a memory, such as the memory 160 and/or the memory 180 in FIG. 2.

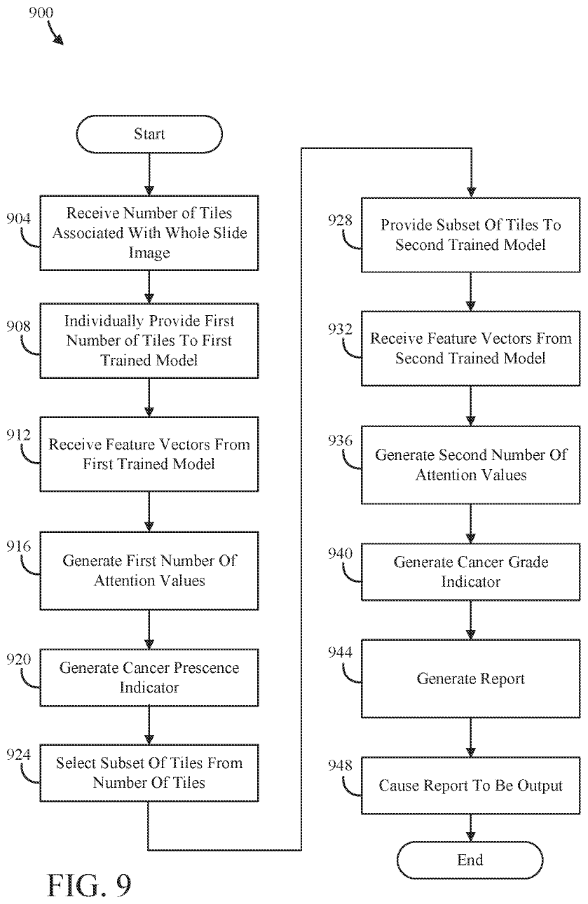

Referring to FIG. 7 as well as FIG. 9, an exemplary process 900 for generating cancer predictions for a patient is shown. The process 900 can be included in the sample image analysis application 132.

At 904, the process 900 can receive number of tiles associated with a whole slide image. The whole slide image can be associated with a patient. In some configurations, the whole slide image can be the whole slide image 704 in FIG. 7. In some configurations, the number of tiles can include a first number of tiles taken at a first magnification level (e.g., 5×) from a whole slide image, and a second number of tiles taken at a second magnification level (e.g., 10× or greater) from the whole slide image. In some configurations, the first number of tiles can include the first number of tiles 708 in FIG. 7. In some configurations, the second number of tiles can include the second number of tiles 740 in FIG. 7. Each of the first number of tiles can be associated with a tile included in the second number of tiles.

At 908, the process 900 can individually provide each of the first number of tiles to a first trained model. In some configurations, the first trained model can be the first trained model 712 in FIG. 7.

At 912, the process 900 can receive feature vectors associated with the first number of tiles from the first trained model. In some configurations, the feature vectors can be the feature vectors 716 in FIG. 7.
At 916, the process 900 can generate a first number of attention values based on the feature vectors associated with the first number of tiles. In some configurations, the process 900 can provide each of the feature vectors to a first attention model. In some configurations, the first attention model can be the first attention model 720 in FIG. 7. The process 900 can receive a first number of attention values from the first attention model. Each attention value can be associated with each tile included in the first number of tiles.

At 920, the process 900 can generate a cancer presence indicator. In some configurations, the process 900 can aggregate instance-level representations into a bag-level feature vector and produce a saliency map that represents relative importance of each tile for predicting slide-level labels. The process 900 can include applying a fully connected layer to the bag-level feature vector in order to generate a cancer presence indicator as described above. In some configurations, the cancer presence indicator can be the cancer presence indicator 732 in FIG. 7.

At 924, the process 900 can select a subset of tiles from the number of tiles. In some configurations, the process 900 can include clustering the number of tiles based on the feature vectors and the first number of attention values. In some configurations, the process 900 can include reducing each feature map associated with each tile to a one-dimensional vector. In some configurations, the process 900 can include reducing feature vectors using PCA to reduce the dimension of the feature vectors. The process 900 can include clustering the final instance feature vectors (i.e., the vectors reduced using PCA) using K-means clustering in order to group similar tiles. In some configurations, the number of clusters can be set to four. The subset of tiles to be used in further processing can be selected based on the number of tiles and the average attention value per cluster as described above.

At 928, the process 900 can provide the subset of tiles to a second trained model. In this way, the subset of tiles can function as the second number of tiles 740 in FIG. 7. In some configurations, the second trained model can be the second trained model 744 in FIG. 7.

At 932, the process 900 can receive feature vectors associated with the subset of tiles from the second trained model. In some configurations, the feature vectors can be the feature vectors 746 in FIG. 7.

At 936, the process 900 can generate a second number of attention values based on the feature vectors associated with the subset of tiles. In some configurations, the process 900 can provide each of the feature vectors to a second attention model. In some configurations, the first attention model can be the second attention model 344 in FIG. 7. The process 900 can receive a second number of attention values from the second attention model. Each attention value can be associated with each tile included in the subset of tiles.

At 940, the process 900 can generate a cancer grade indicator. In some configurations, the process 900 can aggregate instance-level representations from the second trained model into a bag-level feature vector (e.g., the second bag-level feature vector 756) and produce a saliency map that represents relative importance of each tile for predicting slide-level labels. The process 900 can include applying a fully connected layer to the bag-level feature vector in order to generate a cancer presence indicator as described above. In some configurations, the cancer presence indicator can be the cancer grade indicator 760 in FIG. 7. In some configurations, the cancer grade indicator 760 can indicate whether or not the whole slide image 704 is indicative of no cancer (i.e., benign), low-grade cancer, high-grade cancer, and/or other grades of cancer.

At 944, the process 900 can generate a report. The report can be associated with the patient. In some configurations, the process 900 can generate the report based on the cancer presence indicator, the cancer grade indicator, the first number of attention values, the second number of attention values, and/or the whole slide image.

At 948, the process 900 can cause the report to be output to at least one of a memory or a display. In some configurations, at 948, the process 900 can cause the report to be displayed on a display (e.g., the display 108, the display 148 in the computing device 104, and/or the display 168 in the supplemental computing device 116). In some configurations, at 948, the process 900 can cause the report to be saved to memory (e.g., the memory 160, in the computing device 104 and/or the memory 180 in the supplemental computing device 116).

The image analysis application 132 can include the process 400 in FIG. 4, the process 500 in FIG. 5, the process 800 in FIG. 8, and/or the process 900 in FIG. 9. The processes 400, 500, 800, 900 may be implemented as computer readable instructions on a memory or other storage medium and executed by a processor.

### Experiment

An experiment to test the performance of the techniques presented above in conjunction with FIGS. 7-9 is now described. The dataset used contained 20,229 slides from prostate needle biopsies from 830 patients pre- or post-diagnosis. Slides are annotated with slide-level labels extracted from their corresponding pathology reports. There are no additional fine-grained annotations at the pixel- or region-level for this dataset. Additionally, no pre-trained tissue, epithelium, or cancer segmentation networks were relied on, and extensive manual curation to exclude slides with artifacts such as air bubbles, pen markers, dust, etc. was not performed. The dataset was randomly divided into 70% for training, 10% for validation, and 20% for testing, stratifying by patient-level GG determined by the highest GG in each patient's set of biopsy cores. This process produced a test set with 7,114 slides from 169 patients and a validation set containing 3,477 slides from 86 patients. From the rest of the dataset, sampled benign (BN), low grade (LG), and high grade (HG) slides were balanced, which resulted in 9,638 slides from 575 patients. Table 3 shows more details on the breakdown of slides.

TABLE 3

| | No. BN | No. GG 1 | No. GG 2 | No. GG 3 | No. GG 4 | No. GG 5 | No. |
|---|---|---|---|---|---|---|---|
| | Slides | Slides | Slides | Slides | Slides | Slides | Patients |
| Train | 3,225 | 3,224 | 1,966 | 648 | 306 | 269 | 575 |
| Validation | 2,579 | 412 | 307 | 95 | 17 | 67 | 86 |
| Test | 5,355 | 807 | 587 | 148 | 129 | 88 | 169 |
| Total | 11,159 | 4,443 | 2,860 | 891 | 452 | 424 | 830 |

Data preprocessing: The majority of regions on WSIs are background. Thus, slides were converted by downsampling at their lowest available magnification compressed in the .sys file into HSV color space and thresholded on the hue channel to produce tissue masks. Morphological operations such as dilation and erosion were used to fill in small gaps, remove isolated points, and further refine tissue masks. Tiles of size 256×256 at 10× were then extracted from the grid with 12.5% overlap. Tiles that contain less than 80% tissue were discarded from analysis. The number of tiles per slide ranges from 1 to 1,273, with an average of 275. To account for stain variability, a color transfer method was used to normalize tiles extracted from the slide. The scanning objective was set at 20× (0.5 μm per pixel). Tiles were downsampled to 5× for the detection stage model development.

VGGl1 with batch normalization (VGGl1bn) was used as the backbone for the feature extractor in the MRMIL model. A 1×1 convolutional layer was added after the last convolutional layer of VGGl1bn to reduce dimensionality and generate k×256×4×4 instance-level feature maps for k tiles. Feature maps were flattened and fed into a fully connected layer with 256 nodes, followed by ReLU and dropout layers. This produced a k×256 instance embedding matrix, which was forwarded into the attention module. The attention part, which generated a k×n attention matrix for n prediction classes, consisted of two fully connected layers with dropout, tanh non-linear activations, and a softmax layer. Instance embeddings were multiplied with attention weights, resulting in an n×256 bag-level representation, which was flattened and input into the final classifier. The probability of instance dropout was set to 0.5 for both model stages.

The feature extractor was initialized with weights learned from the ImageNet dataset. After training the attention module and the classifier with the feature extractor frozen for three epochs, the last three VGG blocks were trained together with the attention module and classifier for ninety-seven epochs. The initial learning rates for the feature extractor were set at 1×10⁻⁵ and 5×10⁻⁵ for the attention module and the classifier, respectively. The learning rate was decreased by a factor of 10 if the validation loss did not improve for the last 10 epochs. The Adam optimizer and a batch size of one was used.

We further extended our MRMIL model for GG prediction. The cross entropy loss weighted by reversed class frequency was utilized to address the class imbalance problem. Hyperpa-rameters were selected using the validation set. Models were implemented in PyTorch 0.4.1, and trained on an NVIDIA DGX-1.

### Evaluation Metrics

As our test dataset contained over 75% benign slides, accuracy (Acc) alone is biased metric for model evaluation. In addition, the AUROC and AP computed from ROC and precision and recall (PR) curves were used, respectively. For cancer grade classification, the Cohen's Kappa (κ) as defined in Equation 5 below was measured:

$$κ = \begin{matrix}{p}_{o} - {p}_{e} \\ 1 - {p}_{e}\end{matrix} \qquad ( 5 )$$

where pₒ is the agreement between observers, also known as the accuracy and pₑ is the probability of agreement by chance. All metrics were computed using the scikit-learn 0.20.0 package.

### Model Visualization

In addition to quantitative evaluation metrics, interpretability is important in developing explainable machine learning tools, especially for medical applications. In order to have a better understanding of our model predictions, t-Distributed Stochastic Neighbor Embedding (t-SNE) of learned bag-level representations was performed for both stage models. Specifically, for each slide, the flattened n×256 feature vector was utilized before being forwarded to the final classification layer. The learning rate of t-SNE was set at 1.5×10², and the perplexity was set at 30.

The saliency map produced by the attention module in the MRMIL model only demonstrated the relative importance of each tile. To further localize discriminative regions within tiles, Gradient-weighted Class Activation Mapping (Grad-CAM) was utilized. Concretely, given a trained MRMIL model and a target class c, the top k tiles with the highest attention weights were first retrieved, which were fed to the model. Assume o_{c} was the model output before the softmax layer for class c, gradients of o_{c} w.r.t activations A^{l}of l-th feature map in the convolutional layer were obtained through backpropa-gation. Global average pooling over m regions was utilized to generate weights that represent the importance of w×h feature maps. Weighted combinations of d dimensional feature maps then determined the attention distribution of m regions for predicting the target class c as defined in Equation 5.

$${θ}_{l}^{c} = \begin{matrix}1 \\ Z\end{matrix} {Σ}_{i ∈ w} {Σ}_{j ∈ h} \begin{matrix}\partial {o}_{c} \\ \partial {A}_{i , j}^{l}\end{matrix} \\ {α}^{c} = ReLU ( {\sum }_{l = 1}^{d} {θ}_{l}^{c} {A}^{l} ) \qquad ( 5 )$$

where Z=w×h is the normalization constant. The ReLU function removed the effect of pixels with negative weights, since they did not have a positive influence in predicting the given class. α^{c} represents obtained “visual explanation maps” for each image.

### Model Comparison

Blue ratio selection. Blue ratio (Br) image conversion, as defined in Equation 2, repeated below, can accentuate the blue channel of a RGB image and thus highlight proliferate nuclei regions.

$$BR = \frac{100 \times B}{1 + R + G} \times \frac{256}{1 + R + G + B} \qquad ( 2 )$$

where R, G, B are the red, green and blue channels in the original RGB image. Br conversion is one of the most commonly used approaches to detect nuclei and select informative regions from large-scale WSIs. To evaluate the attention-based ROI detection, the first stage cancer detection model was replaced with the Br conversion to select the top q=25% tiles with highest average Br values, referred to as br selection.

Without instance dropout: In this experiment, denoted as w/o instance dropout, whether or not instance dropout could improve the integrity of learned attention map and lead to better performance was investigated.

Attention-only selection: Instead of selecting informative clusters, only the attention map was utilized by choosing the top q=25% tiles with the highest attention values as the input for the second stage model in the att selection experiment.

### Results

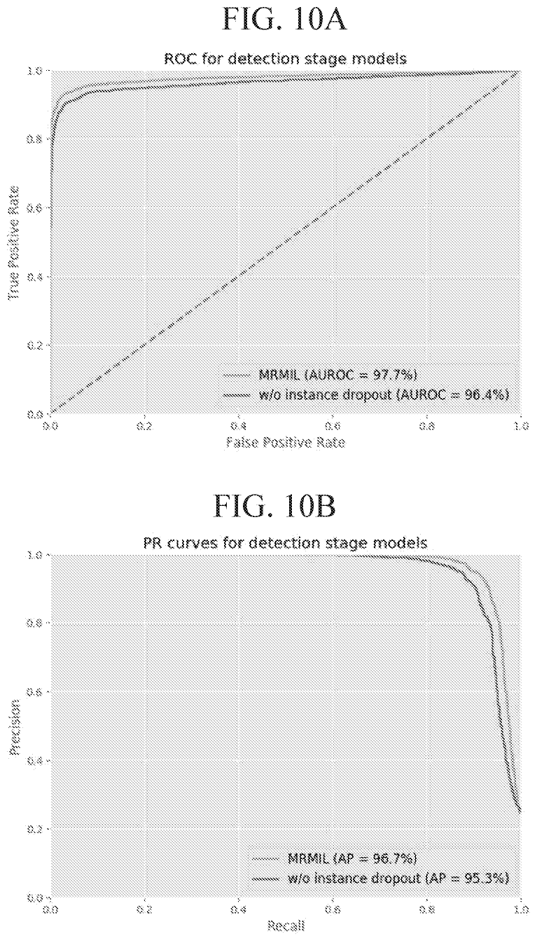

FIG. 10A shows a graph of ROC curves for the detection stage cancer models trained at 5×. FIG. 10B shows a graph of PR curves for the detection stage cancer models trained at 5×. The detection stage model in the MRMIL obtained an AUROC of 97.7% and an AP of 96.7%. The model trained without using the instance dropout method yielded a slightly lower AUROC and AP.

Since our dataset does not have fine-grained annotations at the region- or pixel-level, generated attention maps were visualized and compared with pen markers annotated by pathologists during diagnosis. Markers were masked out as mentioned above, thus they were not utilized for model training.

To further localize suspicious regions within a tile and better interpret model predictions, Grad-CAM was applied on the first detection stage MIL model. Grad-CAM maps were generated for not only true positives (TP), but also false positives (FP) to understand which parts of the tile led to false predictions. Three tiles with highest attention weights were selected from each slide for visualization.

The MRMIL model projects input tiles to embedding vectors, which are aggregated and form slide-level representations. The t-SNE method enables high dimensional slide-level features to be visualized at a two dimensional space.

Table 4 shows model performances on BN, LG, HG classification. The proposed MRMIL achieved the highest Acc of 92.7% and κ of 81.8%. The br selection that relied on the Br image for tile selection only obtained an Acc of 90.8% and a κ of 76.0%. The w/o instance dropout model, got roughly 4% lower κ and 2% lower Acc compared with the MRMIL model. In addition, LG and HG predictions from the classification model were combined and computed the AUROC and AP for detecting cancerous slides. For instance, by zooming in on suspicious regions identified by the detection stage model, the MRMIL achieved an AUROC of 98.2% and an AP of 97.4%, both of which are higher than the detection stage only model.

TABLE 4

| | | | | Cancer |
|---|---|---|---|---|
| | | BN, LG, HG Classification | | Detection |
| Experiment | Model | Cohen's Kappa | Acc | AUROC |
| Name | Details | (%) | (%) | (%) |
| br selection | Multi- | 76.0 | 90.8 | 95.9 |
| | resolution + | | | |
| | Br | | | |
| w/o instance | Multi- | 77.3 | 91.0 | 97.3 |
| dropout | resolution + | | | |
| | Att | | | |
| att selection | Multi- | 80.7 | 92.4 | 98.4 |
| | resolution + | | | |
| | Att + | | | |
| | instance | | | |
| | dropout | | | |
| MRMIL | Multi- | 81.8 | 92.7 | 98.2 |
| | resolution + | | | |
| | Att + | | | |
| | instance | | | |
| | dropout + | | | |
| | clusters | | | |

Using attention maps to select higher resolution tiles improved the κ of the one with br selection by 1%. Instance dropout further boosted the κ by over 3%. The final model MRMIL with all components achieved the highest κ for BN, LG, and HG classification, 98.2% AUROC for detecting malignant slides, and a quadratic κ of 86.8% for GG prediction, which is comparable to state-of- the-art models that require pre-trained segmentation networks.

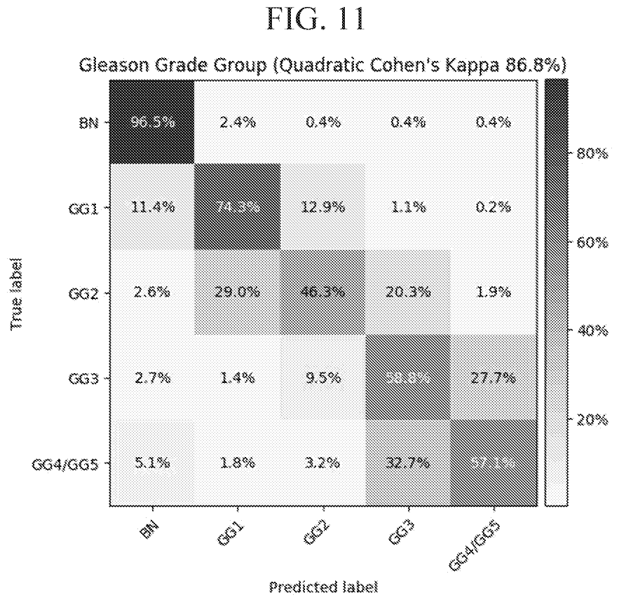

FIG. 11 is a confusion matrix for the MRMIL model on GG prediction. The MRMIL model obtained an accuracy of 87.9%, a quadratic κ of 86.8%, and a κ of 71.1% for GG prediction.

Thus, the present disclosure provides systems and methods for automatically analyzing image data.

The present invention has been described in terms of one or more preferred configurations, and it should be appreciated that many equivalents, alternatives, variations, and modifications, aside from those expressly stated, are possible and within the scope of the invention.

1. A image analysis system comprising:
a storage system configured to have image tiles stored therein;
at least one processor configured to access the storage system and configured to:
access image tiles associated with a patient, each tile comprising a portion of a whole slide image;
individually provide a first group of image tiles to a first trained model, each image tile included in the first group of image tiles having a first magnification level;
receive a first set of feature objects from the first trained model in response to providing the first group of image tiles to the first trained model;
cluster feature objects from the first set of feature objects to form a number of clusters;
calculate a number of attention scores based on the first set of feature objects, each attention score being associated with an image tile included in the first group of image tiles;
select a second group of tiles from the number of image tiles based on the clusters and the attention scores, each image tile included in the second group of image tiles having a second magnification level;
individually provide the second group of image tiles to a second trained model;
receive a second set of feature objects from the second trained model in response to providing the second group of image tiles to the second trained model;
generate a cancer grade indicator based on the second set of feature objects from the second trained model; and
cause the cancer grade indicator to be output to at least one of a memory or a display.

2. The system of claim 1, wherein the second magnification level is greater than first magnification level.

3. The system of claim 1, wherein the whole slide image forms a digital image of a biopsy slide.

4. The system of claim 3, wherein the digital image comprises at least one hundred million pixels.

5. The system of claim 1, wherein the cancer grade indicator includes at least one of benign, low-grade cancer, or high-grade cancer.

6. The system of claim 1, wherein the first trained model comprises a first convolutional neural network, the second trained model comprises a second convolutional neural network, and the second convolutional neural network is trained based on the first convolutional neural network.

7. The system of claim 1, wherein the first trained model and the second trained model are trained based on slide-level annotated whole slide images.

8. The system of claim 1, wherein the at least one processor is further configured to:
generate a report based on the cancer grade indicator; and
cause the report to be output to at least one of the memory or the display.

9. The system of claim 1, wherein the processor is configured to cluster feature objects form the first set of feature objects using k-means clustering.

10. The system of claim 1, wherein the feature objects from the first set of feature objects are feature maps.

11. The system of claim 1, wherein the feature objects of the first set of features objects are feature vectors generated by performing principal component analysis on feature maps.

12. The system of claim 1, wherein the storage system is configured to receive the image tiles from one of a pathology system, a digital pathology system, or an in-vivo imaging system.

13. A image analysis method comprising:
receiving pathology image tiles associated with a patient, each tile comprising a portion of a whole pathology slide;
providing a first group of image tiles to a first trained learning network, each image tile included in the first group of image tiles having a first magnification level;
receiving first feature objects from the first trained learning network;
clustering the first feature objects to form a number of clusters;
calculating a number of attention scores based on the first feature objects, wherein each attention score is associated with an image tile included in the first group of image tiles;
selecting a second group of tiles from the number of image tiles based on the clusters and the attention scores, wherein each image tile included in the second group of image tiles has a second magnification level that differs from the first magnification level;
providing the second group of image tiles to a second trained learning network;
receiving second feature objects from the second trained learning network;
generating a cancer grade indicator based on the second feature objects from the second trained learning network; and
outputting the cancer grade indicator to at least one of a memory or a display.

14. The method of claim 13, wherein the second magnification level is greater than first magnification level.

15. The method of claim 13, wherein the whole slide image is a digital image of a biopsy slide taken from the patient.

16. The method of claim 15, wherein the digital image comprises at least one hundred million pixels.

17. The method of claim 13, wherein the cancer grade indicator includes at least one of benign, low-grade cancer, and high-grade cancer.

18. The method of claim 13, wherein the first trained learning network comprises a first convolutional neural network, the second trained learning network comprises a second convolutional neural network, and the second convolutional neural network is trained based on the first convolutional neural network.

19. The method of claim 13, wherein the first trained model and the second trained model are trained based on slide-level annotated whole slide images.

20. The method of claim 13, further comprising:
generating a report based on the cancer grade indicator; and
delivering the report to at least one of the memory or the display.

21. The method of claim 13, wherein clustering the first feature objects comprises performing k-means clustering on the first feature objects.

22. The method of claim 13, wherein the first feature objects are feature maps.

23. The method of claim 13, wherein the first feature objects are feature vectors generated by performing principal component analysis on feature maps.

24. A whole slide image analysis method comprising:
operating an imaging system to form image tiles associated with a patient, each tile comprising a portion of a whole slide image;
individually providing a group of image tiles to a first trained model, each image tile included in the first group of image tiles having a first magnification level;
receiving a first set of feature objects from the first trained model;
grouping feature objects in the first set of features objects based on clustering criteria;
calculating a number of attention scores based on the feature objects, each attention score being associated with an image tile included in the first group of image tiles;
selecting a second group of tiles from the image tiles based on grouping of the feature objects and the attention scores, each image tile included in the second group of image tiles having a second magnification level that differs from the first magnification level;
providing the second group of image tiles to a second trained model;
receiving a second set of feature objects from the second trained model;
generating a cancer grade indicator based on the second set of feature objects;
generating a report based on the cancer grade indicator; and
causing the report to be output to at least one of the memory or the display.
